(19) United States
(12) Patent Application Publication
(10) Pub. No.: US 2009/0254006 A1
(43) Pub. Date: Oct. 8, 2009

(54) ULTRASOUND ASSISTED TISSUE WELDING METHOD

(76) Inventor: Eilaz BABAEV, Minnetonka, MN (US)

Correspondence Address: Bacoustics, LLC, 5929 BAKER ROAD, SUITE 470, MINNETONKA, MN, 55345, US

(21) Appl. No.: 12/062,547

(22) Filed: Apr. 4, 2008

(51) Int. Cl. A61N 7/00; A61B 18/02; A61F 13/00

(52) U.S. Cl. 601/2; 602/43; 606/21

(57) ABSTRACT

The present invention relates to ultrasound assisted tissue welding, more particularly, to a method and device utilizing ultrasound energy for effective closure and sealing of surgical incisions or other wounds. The device of the present invention comprises an ultrasound generator, an ultrasound transducer, a transducer tip at the distal end of the ultrasound transducer, and a radiation surface. A web such as gauze is placed over the incision. In an embodiment, the device may be used to bring the edges of the tissue to be sealed together as the device applies energy to the tissues. Ultrasonic waves emanating from the radiation surface pass through the web and seal the incision creating a continuous seam. The device may be used with or without additional adhesive materials or therapeutic agents.

### BACKGROUND OF THE INVENTION

The present invention relates to ultrasound assisted tissue welding, more particularly, to a method and device utilizing ultrasound energy for effective closure and sealing of surgical incisions or other wounds.

A surgeon in the course of a procedure is concerned with the repair of damaged tissues. Restoring tissue and circulation integrity is critical to the positive outcome of a procedure regardless of whether the damage was the result of trauma or from the incision necessary to conduct the surgical procedure itself. The oldest method of joining damaged tissues is the use of mechanical devices such as clamps, staples or sutures. Staples, clamps and sutures have a variety of limitations. They require significant skill and are slow to apply. They only effectively seal at the distinct points of attachment. Further, they are ineffectual in a number of highly vascularized organs such as the liver, lung and brain. In addition, they often leak along the line of joinder and cause additional irritation, stress and trauma to surrounding tissue particularly at the points of attachment.

Past efforts have focused on the use of an adhesive or glue capable of bonding tissue surfaces together rapidly while promoting or at least not inhibiting normal healing. Options for tissue adhesives include collagen, albumin and fibrin-based adhesives which may contain a concentrate of fibrinogen and thrombin. Glues based on gelatin cross-linked with an aldehyde have been used, but with limited success. Representative of this class of glues are gelatin-resorcinol cross-linked with formaldehyde or glutaraldehyde. Adhesives such as barnacle glue have been used but are hampered by the ability to purify appreciable quantities of such materials, as well as persistent concerns about the triggering of an immune response. Additional effort has been directed towards finding a suitable synthetic composition operative as a tissue glue. To this end, cyanoacrylates, polyurethanes, polymethylmethacrylates, among other synthetic polymers, have been investigated as tissue glues. Each of these synthetic compositions has had limited success owing to a variety of problems such as toxic degradation products, poor mechanical properties, problems associated with curing, and not being biodegradable.

Among prior art, a pliers-like ultrasound assisted welding device has been developed which utilizes high intensity, high frequency ultrasound and requires access to both sides of the skin tissue. Laser light induced tissue glue curing has also been found to be only partly successful. Laser associated tissue repair has been met with limited success owing to transmural thermal injury and the need for a highly skilled and well equipped surgical team. In view of the enormous development efforts that have taken place, there are few available incision closure methods that meet the requirements of sufficient mechanical strength, biocompatibility and bioavailability, in addition to handling properties and methodologies consistent with a variety of surgical settings.

### BRIEF SUMMARY OF THE INVENTION

The present invention is directed towards a method and device for sealing and closing surgical incisions and other tissue wounds. The invention uses low intensity ultrasound that may include low frequency or high frequency embodiments to repair skin wounds as well as to repair wounds in internal organs that may occur from injury or as a result of incisions produced from surgical procedures such as removal of tumors or diseased tissue. The invention is also appropriate and advantageous for use in veterinary medicine on animal tissues.

Application of energy to tissues to close tissue wounds may sometimes be referred to as “tissue welding.” Tissue welding methods of the present invention may or may not be performed using a glue, a tissue soldering material or other foreign material such as an adhesive. Examples of tissue solders or adhesives which may be used according to the present invention include, but are not limited to; albumin, collagen, fibrin, autologous blood, cyanoacrylates, mussel byssus adhesives, polymer hot melt adhesives and the like.

The device applies ultrasound energy to a wound surface to inactivate and/or destroy infectious agents that may be present in a wound, deliver a medication, and/or sterilize a wound. The device of the present invention comprises an ultrasound generator, an ultrasound transducer, a transducer tip at the distal end of the ultrasound transducer, and a radiation surface.

In its preferred embodiment, the device will bring the edges of the tissue would together as the device applies energy to the tissues. Alternatively the edges of the tissue wound may be held together with sutures, adhesive tape or other means. A web, which by example may include a gauze strip, a film, membrane or other porous or nonporous material, may be soaked in saline or a therapeutic agent. The web is then placed over the incision area. Alternatively, the saline or therapeutic agent may be applied to the wound or the web before the web is placed on the wound. Ultrasonic waves emanating from the radiation surface of the present invention pass through the web, gauze or film layer. As the ultrasound waves pass through the web, the therapeutic agent and/or adhesive agent is pushed into the tissue to be treated.

The invention may be used with or without additional adhesive materials. Generally, energy application alone may act to denature collagen in body tissues. If the tissues are apposed during denaturation and/or while the collagen in the tissues is allowed to renature, the collagen in once-separated tissues binds together to bring and hold the tissues together without additional adhesives.

The invention as described in the specification, drawings and claims provides several advantages over prior art methods and devices.

One advantage of the invention is that it may be used to bring the edges of an incision together as it seals the incision.

Another advantage of the invention is that it seals to form a continuous seam at the incision line.

Another advantage of the invention is that it can be used with available access from only one side of the tissue, for example, the epidermal side of skin tissue.

Another advantage of the invention is that it may be used to cool the surface of the wound.

Another advantage of the invention is that it seals to form a flat seam with scar tissue at the incision line with or without sutures or other mechanical means.

Another advantage of the invention is that it may be used to seal wounds on a variety of organ tissues.

Another advantage of the invention is that it may be used with or without glues or other sealants.

Another advantage of the invention is that it may be used with or without therapeutic agents.

Another advantage of the invention is that the applied ultrasound can have an antimicrobial effect for the treated and surrounding tissue.

Another advantage of the invention may be that ultrasonic energy applied as described provides inherent pain relief effect resulting from the application of ultrasonic energy.

Another advantage of the invention is that it allows surgeons to repair incisions in a time efficient and cost effective manner and minimize post operative side effects to the patient.

### BRIEF DESCRIPTION OF THE SEVERAL VIEWS OF THE DRAWING

FIG. 1 depicts a dimensional view of the apparatus according to the present invention.

FIG. 2 depicts a cross-sectional view of an embodiment of the transducer tip as radiation is delivered to the tissue surface.

FIG. 3 depicts a top plan view of the transducer tip.

FIG. 4 depicts a dimensional view of an embodiment showing use of the radiation surface for tissue welding.

FIGS. 5A-5J shows dimensional views of several embodiments of the radiation surfaces to be used for tissue welding.

### DETAILED DESCRIPTION OF THE INVENTION

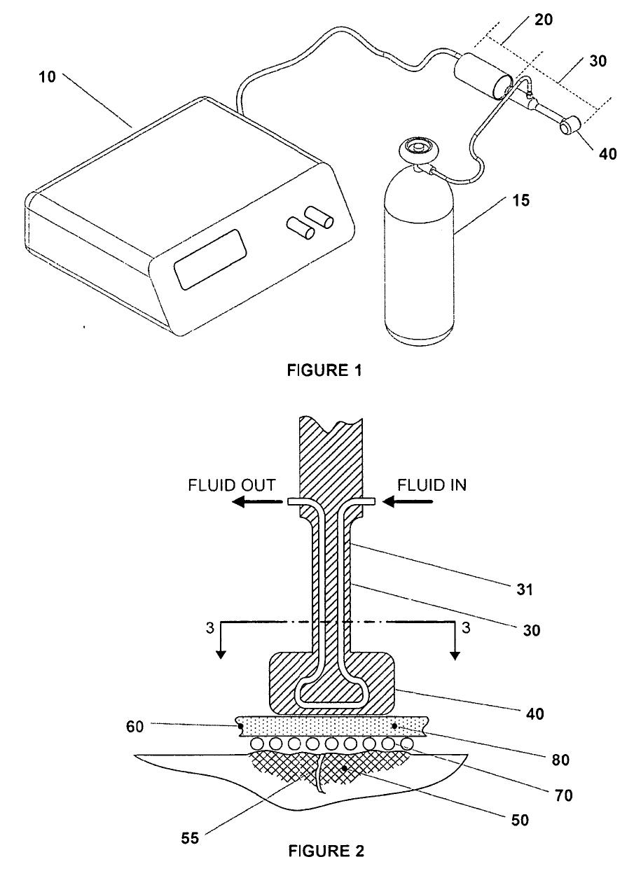

A general view of an embodiment of the present apparatus is shown in FIG. 1. The present invention relates to devices and methods for the use of ultrasonic energy to closing and sealing wounds. The invention is applicable to wounds resulting from surgical incisions on skin as well as incisions on internal organs. Highly controllable, precise delivery of ultrasonic energy allows optimal sealing of tissue wounds without damaging surrounding tissue.

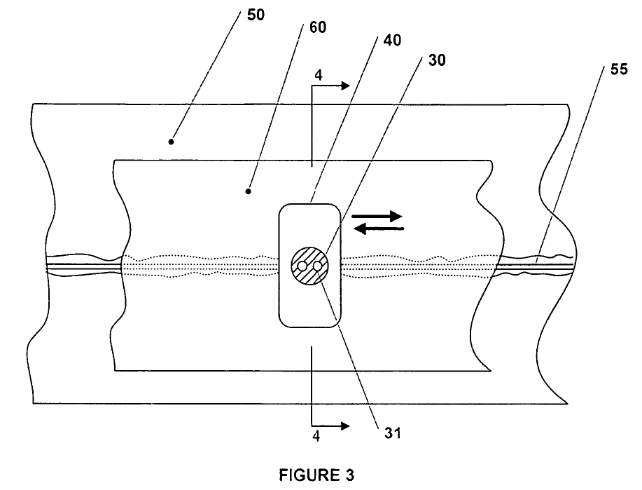

FIGS. 1-5 relate generally to describe the invention in some of its embodiments. The apparatus of the present invention may be a hand held device with a housing surrounding an ultrasound transducer 20 as shown in FIG. 1. The housing provides a surface for the surgeon to hold for manipulation of the device over the wound. The housing also may provide dampening and isolation so that the heat, electrical and mechanical energy emitted from the ultrasound transducer 20 does not reach the operator of the device. The ultrasound transducer 20 is driven by an ultrasound generator 10. The ultrasound generator 10 is typically powered with standard AC current which is electrically connected to an ultrasound transducer 20 through a cable and activated with a hand or foot operated switch. The ultrasonic transducer is pulsed according to a driving signal generated by the ultrasound generator 10 and transmitted to die ultrasonic transducer by cable. The driving signals as a function of time may be rectangular, trapezoidal, sinusoidal, or other signal types as would be recognized by those skilled in the art.

The ultrasound generator 10 may also be programmable to provide a rapid pulsed on-off signal to the ultrasound transducer 20 to control and limit temperature rise within the tissue. This pulsed signal may vary between 0 to 100% depending on the application.

The distal end of the ultrasound transducer 20 is attached to a transducer tip 30 for conditioning and directing the ultrasonic energy toward the tissue area selected for treatment. The ultrasound waves emitted have a frequency and amplitude. The ultrasonic frequency may be used in embodiments that include low frequency or high frequency embodiments that operate within the range of 16 kHz and 20 mHz. The amplitude of the ultrasonic waves may be between 1 micron and 250 microns with a preferred amplitude in the range of 10 to 50 microns and a recommended amplitude of 20 microns.

The ultrasound transducer tip 30 may contain one or more interior chambers for passage of a cryogenic fluid 15. The ultrasonic tip 30 may also contain one or more temperature sensors which may control the flow rate of cryogenic fluid 15 through the ultrasound transducer tip 30 to maintain a consistant preselected temperature at the tip regardless of the ultrasound energy emitted from the tip.

Those skilled in the art will recognize that the ultrasound tip 30 can be a single piece unit or composed of one or more individual separate pieces that are detachable from the device. This allows interchangeability of portions of different embodiments of the tip as well as easier cleaning/sterilization of portions of the device and/or allows construction of disposable single-use portions of the ultrasound transducer tip 30. The transducer tip 30 is typically made from a metal such as alloys of titanium, aluminum and/or stainless steel. The portions of the ultrasound transducer tip 30 may also be made from plastic for disposable single-use embodiments of selected portions or protective coverings of the transducer tip 30.

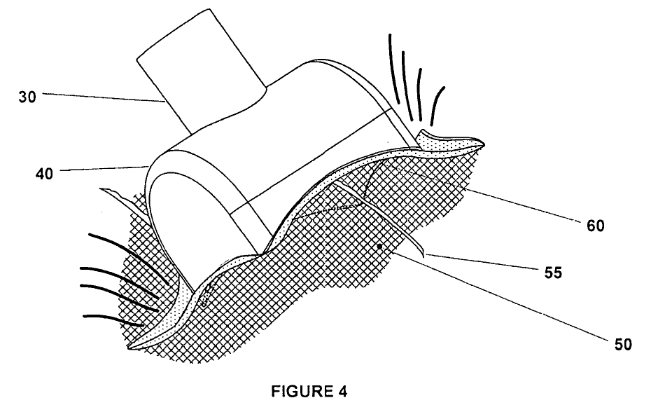

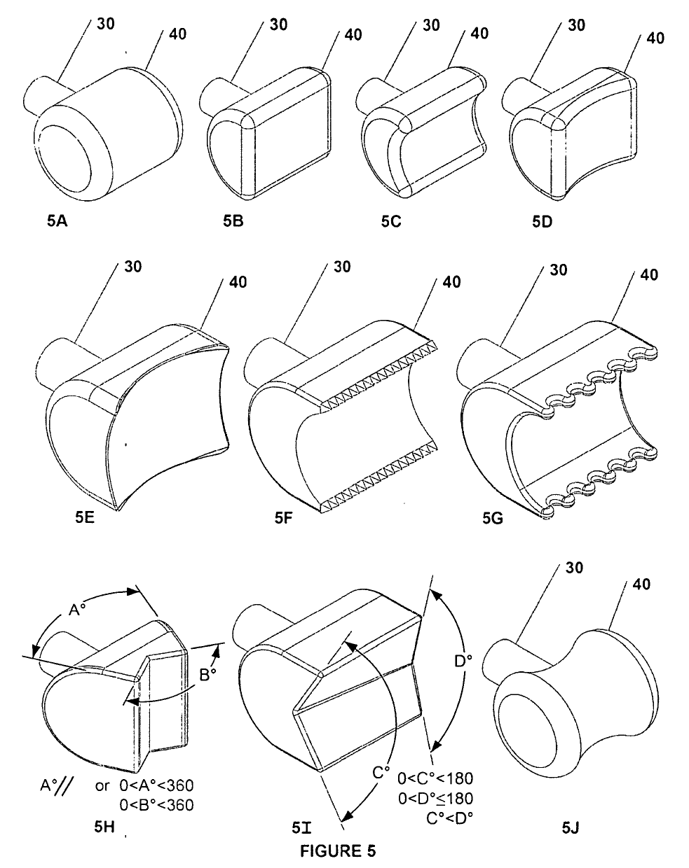

In other aspects, the distal end of the ultrasonic tip may have various sizes and geometric shapes of the radiation surface 40 such as flat, concave, convex, rounded, ridged and/or waved. Some of these embodiments are shown in FIGS. 5A-5J. FIG. 5A shows a convex cylindrical embodiment. FIGS. 5C, 5D, 5E, 5F, 5G and 5J show various concave embodiments of the radiation surface 40 forming a channel or interior portion. FIG. 5B shows a planar embodiment of the radiation surface 40. FIGS. 5H and 5I show embodiments with the radiation surface 40 forming a triangular channel or interior portion with inner and outer walls. FIG. 5H shows a radiation surface with constant angular dimensions for the inner and outer walls. FIG. 5I shows a radiation surface 40 with inner walls having a variable angular dimension creating a variable depth channel and the outer walls being parallel to each other. This embodiment offers advantages for bringing the edges of the incision 55 together for improved incision closure. The inner wall variable angular dimension could also be set at a constant channel depth to form a triangular shaped radiation surface 40 with a variable channel width.

The edge surfaces of the radiation surface 40 may be rounded edge, sharp edged, scalloped or serrated using a various combinations of shapes. FIG. 5F shows an edge constructed of a plurality of triangular shaped serrations. FIG. 5G shows the wave shaped embodiment of the edge. The various shapes can be chosen as appropriate for various procedures to be performed on the tissue. The preferred embodiment includes a concave center portion traversing a length of the radiation surface as shown in FIGS. 3-5. A detachable ultrasound transducer tip 30 can allow a surgeon to vary the geometric shape of the distal end as appropriate either between procedures or during the course of a procedure.

As shown in FIG. 2, preferably a web 60, gauze, membrane or film is placed over the incision 55 or wound opening. The web 60 is preferably a gauze strip, but may include any appropriate porous material or a solid material such as a plastic sheet. The web 60 is preferably wetted with a liquid or gel fluid which may be a saline solution, an adhesive 70 material or a therapeutic 80 solution. The liquid or gel provides a coupling medium between the radiation surface 40 for improved ultrasound transmission between the radiation surface 40 and the tissue 50. Using the web 60 isolates the radiation surface 40 from the wound surface which may reduce heat and trauma impacts on the tissue and eliminate any friction from direct contact between the vibrating radiation surface 40 and the tissue 50.

Furthermore, the porosity of the gauze from the presence of openings in the structure of the web 60 enhance the ability of the ultrasound waves to move into the wound as well as transport the adhesive 70 material and/or therapeutic solution into the Wound area. The web 60 thickness as well as the type of material can be varied to enhance the effect of the invention upon treating the incision. This occurs due to the different transmissive properties of materials that may be used and the fact that the distance of the radiation surface 40 from the tissue 50 changes as a result of varying web 60 thicknesses. Doing so will allow different portions of the ultrasound standing wave generated at the radiation surface 40 to interact on different depths of the tissue 50 being treated. The thickness of the web 60 will affect the characteristics of the ultrasound that is emitted from the radiation surface 40 that reaches the tissue 50. In addition, increasing the thickness of the web 60 will increase the amount of fluid that can be applied to a given area of tissue.

In the preferred embodiment as shown in FIGS. 3-5, the invention may be used to bring the edges of an incision 55 together as it seals the incision. This may be accomplished by placing the web 60 over the two free edges of the incision opening) and pressing the radiation surface 40 against the web 60 at one end of the incision 55. The radiation surface 40 is then held against the web 60 to assert pressure on the tissue 50 as the tip is pushed along the length of the incision line. Preferably the device is held so that the longitudinal axis of the transducer tip 30 forms an angle between 0 and 90 degrees with the longitudinal plane defined by the surface of the wound. Under the preferred embodiment, a radiation surface 40 will have a concave parabolic shape along the length of a center line of the radiation surface. This configuration is advantageously used so that the pressure exerted on the tissue 50 is directed toward the edges of the radiation surface 40 as they contact the tissue 50. The edges then may then tend to grab and urge the tissue toward the concave area along the center line of the radiation surface 40. The free edges of the tissue 50 are pushed together as the transducer tip 30 moves along the incision line. This results in a continuous tight seam behind formed as ultrasound radiation is applied to the incision line.

The radiation surface 40 in its concave embodiment can be designed so that the ultrasound waves are focused along the center line of the wound seam. This configuration will enhance energy input at the incision line itself. The gathering of the edges of the wound opening, together, as well as the continuous seam formed from this method results in a flat seam when the procedure is completed and greatly reduces and may eliminate scarring from the wound or incision opening.

As is apparent from the description of the methods of use of this invention, all access to tissue 50 is only required from one side of the tissue surface. For example, skin wounds, may be performed with access only from the epidermal side of the skin tissue being required. The device does not require access from beneath the skin surface to perform its function.

The invention allows simultaneous closing and sealing of the incision opening in a single step procedure without the use of sutures, staples, clamps or adhesive strips. Alternatively, the incision opening may be initially closed with sutures, staples or adhesive strips before the web 60 layer or gauze is placed and the ultrasound is applied. The method of the invention may provide a continuous seam and removes stress points associated with sutures or like methods.

When access to at least one surface of the tissue wound site is available, such as during open surgical procedures, the invention may be used to seal wounds on organ tissue such as heart, liver, brain, stomach and lung in addition to skin incisions using the described techniques of the invention. The invention is particularly helpful for use on internal organ tissues which are difficult to suture as a result of their characteristic properties, such as lack of rigidity, tendency to leak, lack of strength to withstand stress at attachment points and severe negative consequences that result if they fail to effectively hold.

The invention may be used with or without glues, adhesives 70 or other tissue sealants, which when used, may be applied in a liquid or gel form to the wound surface, the web 60 surface or allowed to soak into porous materials such as gauze and applied to the wound with the gauze.

The invention may be used with or without additional adhesive materials. Use of the inventive device may be used to rely on energy application alone to denature collagen in body tissues. If the tissues are apposed during denaturation and/or while the collagen in the tissues is allowed to renature, the collagen in once-separated tissues binds together to bring and hold the tissues together without the use of additional adhesives in the medical procedure.

Adhesives 70 may be comprised of fiber forming proteins such as, but not limited to: collagen, elastin, as well as glycoproteins associated with extracellular matrices such as, but not limited to: laminin, entactin, and/or fibronectin. The invention may also be used with adhesives 70, other glues and sealants known in the art for tissue repair. When used with adhesives, the vibration, cavitation and stimulation of the ultrasound enhance performance of the adhesive creating a stronger bond than can be achieved without the application of ultrasound. The cavitation, motion and energy input allow a stronger bond between tissue structure as well as between the tissue 50 and adhesive 70. The ultrasound will also enhance curing of the adhesive 70. The combined effects greatly reduce the risk of incision repair failure.

The invention may also be used with or without therapeutic agents 80. Therapeutic agents 80 may be added in the same manner as the adhesives 70, such as directly to the wound, directly to the web 60 or allowed to soak into the web 60 and applied with the web 60. Therapeutic agents 80 may include saline and/or various drugs and medications. Typical examples of therapeutic agents 80 include antibiotics, analgesics, anesthetics, pain relief medications, moisturizers, tissue growth enhancers, and blood clotting promoters. Other topical ointments that may be used include vitamin A, D, and E ointments, silicone gel and/or various lipid creams.

Ultrasound stimulation of tissue, particularly nerve cells, is known to mitigate pain. This property of ultrasound can be used advantageously by the surgeon to make the process less painful due to the pain relief effect resulting from the application of ultrasonic energy. This property can also be used synergistically with analgesics or other pain relief agents that may be topically applied or with other means.

In addition to the enhanced pain relief associated with ultrasonic assisted application of the therapeutic agents 80, the application of ultrasonic energy itself makes the treated area less painful due to the pain relief provided by the application of the energy to nerve endings associated with the wound area. The shape of the radiation surface can be modified to optimize this effect.

Under the preferred embodiment, the cryogenic cooling of the ultrasound tip also has therapeutic value associated with wound treatment. The cryogenic fluid 15 used to cool the transducer tip 30 will also cool the surface of the wound. Cooling an incision wound is common practice to reduce the edema, pain, swelling and/or inflammation associated with wound treatment. The cryogenic fluid 15 may be such gases or liquids as would be recognized by those skilled in the art such as, for example, liquid nitrogen. The cryogenic fluid 15 may be delivered to the interior passage 31 from a source through a cryogenic fluid delivery tube which is attached to the transducer tip 30. Thereby, the feature for removing heat generated in the ultrasound transducer tip 30 may be utilized for therapeutic affect by also cooling the tissue 50. The invention may include a temp feedback/sensor located in the transducer tip 30 to maintain proper temperatures at the radiation surface 40.

The application of ultrasonic energy may have an antimicrobial effect for the treated and surrounding tissue. The application of ultrasound energy is known to produce cellular disruption and microbial inactivation due to cavitation in gases, liquids and/or tissues to which it is applied. The cavitations and the ultrasound energy are able to inactivate microbes in the area of treatment through cellular disruption, denaturization and other means. This effect can reduce the chance of infection, thereby greatly enhancing patient recovery, since post surgical infection can be a major consideration in optimal patient recovery.

The procedure according the present invention may be rapidly and economically performed as compared to prior art procedures. The uniformity associated with having a continuous seam with no discrete points of stress promotes strength of the incision repair as described. Furthermore, the resulting uniformity of the seam can reduce or eliminate the problems associated with scarring. The controlled conditions under which the procedure is performed reduce the possibility of disfiguring wound healing that may result from sutures and the like.

Although specific embodiments have been illustrated and described herein, it will be appreciated by those of ordinary skill in the art that any arrangement that is calculated to achieve the same purpose may be substituted for the specific embodiments shown. It is to be understood that the above description is intended to be illustrative and not restrictive. Combinations of the above embodiments and other embodiments will be apparent to those having skill in the art upon review of the present disclosure. Although method steps may be presented in a particular order, the order is intended to be illustrative and not restrictive. The scope of the present invention should be determined with reference to the appended claims, along with the full scope of equivalents to which such claims are entitled.

I claim:

1. A method for sealing a wound comprising the steps of:
placing a web over an incision;
contacting the web with a radiation surface portion of a transducer tip;
bringing at least two edges of the wound together;
propagating ultrasonic energy through the web into the wound; and
sealing the wound.

2. The method of claim 1 having an interior portion on the radiation surface.

3. The method of claim 2 wherein the step of bringing at least two edges of the wound together use the radiation surface to urge the edges together.

4. The method of claim 1 wherein the step of bringing at least two edges of the wound together uses sutures to bring the edges together.

5. The method of claim 1 having the radiation surface being tilted at an angle between 0 degrees and 90 degrees from a plane defined by the wound surface.

6. The method of claim 1 having the additional step of passing a cryogenic fluid through an interior passage to cool the transducer tip.

7. The method of claim 6 having the additional step of controlling the cryogenic fluid with a temperature sensor.

8. The method of claim 1 having the additional step of passing a cryogenic fluid through an interior passage to cool the wound.

9. The method of claim 1 wherein the ultrasound energy is generated from a signal form selected from the group including sinusoidal, rectangular, trapezoidal or triangular.

10. The method of claim 1 wherein the web is a gauze.

11. The method of claim 1 having the additional step of saturating the web with a therapeutic agent.

12. The method of claim 1 wherein the ultrasonic energy carries the therapeutic agent into the wound.

13. The method of claim 1 having the additional step of saturating the web with a saline solution.

14. The method of claim 1 having the additional step of applying an adhesive to the wound before propagating the ultrasonic energy.

15. The method of claim 1 having the additional step of deactivating microbes with the ultrasonic energy.

16. The method of claim 1 having the additional step of directing the radiation surface by holding a housing.

17. The method of claim 1 wherein the radiation surface emits ultrasound waves at a wavelength between 16 kHz and 20 mHz.

18. The method of claim 1 wherein the radiation surface emits ultrasound waves at an amplitude between 1 micron and 250 microns.

19. The method of claim 1 having the additional step of providing pain relief with the ultrasonic energy.

20. The method of claim 1 wound consists of tissue selected from the group of heart, lung, liver, brain and stomach.
